(19) United States
(12) Patent Application Publication
(10) Pub. No.: US 2017/0023583 A1
(43) Pub. Date: Jan. 26, 2017

(54) MULTIPLEX ENZYME ASSAY USING MASS SPECTROMETER-BASED FLOW CYTOMETER

(71) Applicant: Fluidigm Canada Inc., Markham (CA)

(72) Inventor: Olga Ornatsky, Richmond Hill (CA)

(21) Appl. No.: 15/039,797

(22) PCT Filed: Nov. 26, 2014
(86) PCT No.: PCT/CA2014/051131

§ 371 Date: May 26, 2016

Related U.S. Application Data
(60) Provisional application No. 61/909,367, filed on Nov. 26, 2013.

(51) Int. Cl. G01N 33/68; G01N 33/58; C07K 1/13; C12Q 1/37

(52) U.S. Cl. CPC G01N 33/6848; C12Q 1/37; G01N 33/58; C07K 1/13; G01N 2333/95; G01N 2458/15

(57) ABSTRACT

The present invention generally relates to methods for the detection of enzymes using elemental analysis.

### FIELD

The present invention generally relates to methods for the detection of enzymes using elemental analysis.

### BACKGROUND

Owing to their role in maintaining animal homeostasis, the assay and pharmacological regulation of enzymes have become key elements in identifying possible therapeutic agents. Proteases are a subclass of protein-degrading enzymes that have recently been shown to play a vital role in signaling pathways, the disregulation of which can result in cancer, cardiovascular disease, and neurological disorders. Of the approximately 400 known human proteases, approximately 14% are being studied as potential drug candidates. Small-molecule inhibitors of proteases are now considered valuable therapeutic leads for the treatment of degenerative diseases, for the treatment of cancer, and as antibacterials, antivirals and antifungals.

There is a need for a robust, sensitive, and quantitative enzyme assay that allows for simultaneous measurement of multiple enzymatic reactions. Such an assay can allow for conservation of valuable biological sample and reagents, achieve high-throughput and reduced assay time, and decrease the overall cost of enzyme analysis.

### SUMMARY

One aspect of the invention is a method for detecting protease activity in a biological fluid. The method comprises attaching a coded bead to a first amino acid of a peptide substrate to form an immobilized peptide substrate, the peptide substrate comprising a first amino acid and a last amino acid and being a substrate for a protease enzyme: attaching an element tag to the last amino acid of the peptide substrate to form a tagged peptide substrate: incubating the immobilized, tagged peptide substrate with the biological fluid: and detecting the element tag and the coded bead in the biological fluid by elemental analysis.

Another aspect of the invention is a method for detecting protease activity in a biological fluid. The method comprises attaching a coded immobilization moiety to the first amino acid of at least five different peptide substrates to form at least five different coded immobilized peptide substrates, the peptide substrates being substrates for different protease enzymes: attaching a different element tag to the last amino acid of each of the at least five different peptide substrates to form tagged peptide substrates: incubating the immobilized, tagged peptide substrates with the biological fluid: and detecting the element tag and the coded immobilization moiety in the biological fluid by mass cytometry.

Other objects and features will be in part apparent and in part pointed out hereinafter.

### DETAILED DESCRIPTION

It has been discovered that elemental analysis, particularly mass cytometry, can be used to enable the rapid, quantitative measurement of enzymatic reactions, including the detection and quantification of multiple enzymatic reactions in a single assay. When carrying out the assay in a multiplexed format, the activities of a plurality of enzymes can be determined. A plurality of enzyme substrates can be combined with a whole cell lysate, under conditions that allow processing of the substrates to products by the corresponding enzymes, such that each enzyme produces a different product. The amount of each product is determined and this amount is an indication of the corresponding enzyme activity.

In certain embodiments, each different enzyme substrate is labeled with a different detectable label to facilitate quantitation of the respective product. Although any suitable detectable label can be used for this purpose, element tagged substrates are particularly suitable for multiplex reactions.

The mixture can be fortified by a candidate effector (i.e., an inhibitor or an activator of enzyme activity). Effectors are described in more detail herein.

There are several advantages of the disclosed assay methods. The primary advantages include the following: the assays provide measurement of multiple enzymes in one reaction: the assays do not use antibodies or radioactive isotopes: the assays are insensitive to light the reagents used in the assays have very long shelf life: the assays do not require purification steps: the assays are amenable to miniaturization and automation: and the assay can be performed in real-time for kinetic studies.

Because of these and other advantages, the assays are achieve high-throughput, versatility, and sensitivity that provides quantitative results that can be used to develop enzyme inhibitor screening methods capable of simultaneously screening many inhibitors.

Another application of the methods provided by the present disclosure lies in the creation of substrate suspension arrays for high-throughput screening of enzymatic activity.

The substrate for a specific enzyme can be tagged with an element-encoded bead and an element tag distal to the site of enzymatic cleavage and contacted with a mixture of enzymes that specifically act on different substrates and the beads can be interrogated sequentially in a cytometric fashion (mass cytometer). For example, a test enzyme can cleave a substrate such that the element tag is released into solution. It is understood that the enzyme present in a test sample will cleave at least a portion of tagged substrate in the sample. Performing the enzymatic reaction for longer times, adjusting pH, adjusting the temperature, and/or increasing enzyme concentration can result in complete or optimal cleavage of the tagged substrate. The presence or absence of the element tag with the coded bead provides a measure of the cleavage of the substrate by the enzyme with the presence of the element tag and the coded bead in a particle indicating that the substrate was not cleaved by the enzyme and the presence of only the coded bead indicating that the substrate was cleaved by the enzyme.

In certain embodiments, an enzyme substrate can be attached to a metal chelate or to a polymer having a metal chelate. Non-limiting examples of suitable metal chelates include a diethylenetriaminepentaacetate (DTPA) ligand or a 1.4.7.1 0-tetranzacyclododecane-1.4.7.1 0-tetraacetic acid (DOTA) ligand. More generally™, one skilled in the art can understand that any suitable chelate with a particular way of binding metal ions and atoms can be used.

Generally, the automated synthesis of peptides can be routinely carried out by one of ordinary skill in the art. For example, the enzyme substrates can be directly synthesized on solid beads in peptide synthesizers (one-bead one-compound library synthesis). The solid beads can be comprised of TENTAGEL™, which is a divinyl benzene cross-linked polystyrene resin that contains poly(ethyleneglycol) (PEG) grafts and is used for solid-phase peptide synthesis (SPPS). Other members of this polymer family include ARGOGEL™, NOVAGEL™, and NOVASYN TG™. Other suitable materials for use in the automated synthesis of peptides are generally known to those of skill in the art.

This method can be used for sensitive quantitative measurement of multiple enzymatic reactions in one tube in real-time as well as the creation of substrate suspension array libraries for enzyme substrate identification and optimization. For this purpose beads coded with different elements or various ratios of several elements can be covalently attached to a specific substrate tagged with an element not present in the beads in such a way that one bead type represents one substrate. Because the number of elements and their stable isotopes that can be used is greater than 50 and each type of bead can be encoded by a unique combination of metals, the number of different specific probes linked to uniquely coded beads can be very large over 10⁶ different coded beads are feasible).

Generally, the processes and methods described herein include at least four steps. In one step, a first amino acid of a peptide substrate is attached to an element-tagged support thereby forming an immobilized peptide substrate. In another step, an element tag is attached to the last amino acid of an immobilized peptide substrate, thereby forming a tagged, immobilized peptide substrate. In a further step, the tagged, immobilized peptide substrate is incubated with a biological medium. And in a farther step, elemental analysis is used to detect the presence of the element tags and/or element-tagged support in the biological medium.

In an initial step of the analytical method, a first amino acid of a peptide substrate is attached to an element-tagged support, thereby forming an immobilized peptide substrate.

Generally, the peptide substrate can be attached to the element-tagged support using any suitable attachment method known to those skilled in the art.

Typically, the first amino acid of the peptide substrate is covalently bonded to the element-tagged support. For example, the surface of the element-tagged support can be functionalized with a reactive chemical group. Non-limiting examples of reactive chemical groups include carboxylate, amino, thiol, epoxy, aldehyde, hydroxyl, sulfhydryl, and hydrazide groups. Free radicals and/or radical cations can be used to initiate the coupling reaction.

For example, the element-tagged support can have a surface which has been functionalized with pyrrole-2.5-dione (maleimido), sulfonic acid anion, or p-(chloromethyl) styrene.

The peptide substrate can also be immobilized using a non-covalent coupling method. For example, the peptide substrate can be physically adsorbed onto the element-tagged support.

Particularly, the peptide substrate can be immobilized on the element-tagged support using a biotin-streptavidin complex. For example, biotin can be linked to the first amino acid of the peptide substrate and streptavidin can be linked to the element-tagged support, or vice versa.

In another step of the analytical method, an element tag is attached to the last amino acid of an immobilized peptide substrate, thereby forming a tagged, immobilized peptide substrate.

Generally, the element tag can be attached to the immobilized peptide substrate using any suitable attachment method known to those skilled in the art.

For example, the element tag can be covalently bonded to the last amino acid of the immobilized peptide substrate. To facilitate covalent bonding, the element tag can include one or more reactive chemical groups. Non-limiting examples of reactive chemical groups include carboxylate, amino, thiol, epoxy, aldehyde, hydroxyl, sulfhydryl, and hydrazide groups. Free radicals and/or radical cations can be used to initiate the coupling reaction.

Alternatively, the element tag can be attached to the immobilized peptide substrate using a non-covalent coupling method. For example, the element tag can be attached to the substrate using a biotin-streptavidin complex. For example, biotin can be linked to the last amino acid of the immobilized peptide substrate and streptavidin can be linked to the element tag, or vice versa.

The tagging and immobilization steps described above can be performed in any order, thereby forming a tagged, immobilized peptide substrate.

In a farther step of the analytical method, the tagged, immobilized peptide substrate is incubated with a biological medium.

As used herein, the term “biological medium” broadly refers to any material that contains, is believed to contain, or may containing an enzyme, an enzyme activator, and/or an enzyme inhibitor. For example, the biological medium can comprise a sample obtained from tissue, fluid, and cells of an animal, plant, fungal, bacterial, or viral origin. Non-limiting examples of samples that can be included within the biological medium include sputum, plasma, urine, peritoneal fluid, pleural fluid, milk, saliva, synovial fluid, amniotic fluid, and extracts from blood cells, tissue and fine needle biopsies.

Additional non-limiting examples of samples that can be included within the biological medium include homogenized model viruses and cell cultures of animal, plant, bacteria, and fungal cells, wherein gene expression states can be manipulated to explore the relationship among genes and to express reporter molecules (e.g., beta-galactosidase).

The biological medium can also include solutions of purified biological molecules, including, for example, proteins, peptides, DNA, RNA, polysaccharides, and lipids. These biological molecules can be natural or recombinant.

A tagged, immobilized peptide substrate can be incubated with the biological medium for a period of time sufficient to allow the enzymes in the biological medium to react with at least a portion of the tagged, immobilized peptide substrate.

For example, if the biological medium contains a protease that is specific to the peptide substrate, the protease can conduct proteolysis on the tagged, immobilized peptide substrate. The proteolysis reaction cleaves the tagged, immobilized peptide substrate into a first portion (comprising a first amino acid attached to the element-tagged support) and a second portion (comprising a last amino acid attached to an element tag).

If desired, a more complete reaction can be obtained by increasing the duration of the incubation, adjusting the pH of the biological medium, adjusting the temperature of the biological medium, and/or increasing enzyme concentration. The optimum pH and temperature of the biological medium will depend upon the particular enzymes that are active, as understood by one of skill in the art.

“Microparticle, microspheres, microbeads, nanobeads, nanoparticles, beads, or particles” are used interchangeably and can denote various sizes and shapes of particles and for the purpose of this invention have similar functionality.

“Element stained particles or particularly lanthanide imbibed particles)” contain a plurality of elements (isotopes), which are used to mark a microsphere. The stain elements are either uniformly diffused throughout the body of said microsphere or penetrate said microsphere in a manner that results in formation of a volume distribution of elements in distinct ways. These latex microspheres can be formed from polystyrene. polymethyl-methacrylate, acrylonitrile, etc. The surface of the particles can be chemically functionalized with carboxyl, amino, hydroxyl, sulfhydryl, hydrazide derivatives or the like. The average size of microspheres can range between 0.3 microns in diameter to 10 microns. Suitable particles are described, for example, in U.S. Application Publication No. 2010/0144056, which is incorporated by reference in its entirety.

Following the incubation step, the immobilized peptide substrate can optionally be separated from the biological medium.

Generally, the immobilized peptide substrate can be separated from the biological medium using chromatographic, centrifugation, filtration, or dialysis methods that are known in the art. For example, AMICON® ULTRA-0.5 centrifugal filter devices can be used for separating small nanobeads (0.3-1.0 microns) from cleaved substrate. In this case, the element-tagged cleaved part of the peptide substrate can be collected from the flow-through of the spin filter in the bottom part, while the immobilized cleaved substrate can be retained in the upper chamber. Multiple washes of the particles can be performed using these devices. Microparticles with immobilized peptide substrate of larger size (1-10 microns) can be subjected to centrifugation at 10,000 G for 10 minutes to achieve complete sedimentation of particles. The liquid on top of the pelleted microparticles will contain the cleaved part of element-tagged peptide substrate.

When the immobilized peptide substrate is separated from the biological medium, the remaining components of the biological medium are referred to as an “analyte solution”. The components of the analyte solution (e.g., the element tags) can be detected, for example, using elemental analysis.

In a farther step of the analytical method, elemental analysis is used to detect the presence of element tags in the biological medium.

Generally, elemental analysis refers to a process where a sample is analyzed for its elemental composition and, optionally, its isotopic composition. Non-limiting examples of elemental analysis methods include optical atomic spectroscopy, such as flame atomic absorption, graphite furnace atomic absorption, and inductively coupled plasma atomic emission, which probe the outer electronic structure of atoms: mass spectrometric atomic spectroscopy, such as inductively coupled mass spectrometry, which probes the mass of atoms; and x-ray fluorescence, particle induced x-ray emission, x-ray photoelectron spectroscopy, and Auger electron spectroscopy, which probe the inner electronic structure of atoms.

As used herein, the term “volume elemental analysis” refers to a process wherein an analyzed sample is interrogated in a manner that detects an average atomic composition over the entire volume of the sample.

As used herein, the term “particle elemental analysis” refers to a process wherein an analyzed sample, composed of solid particles dispersed in a liquid, is interrogated in such manner that the atomic composition is recorded for individual particles. An example of particle elemental analysis is mass cytometry, wherein the analytical instrument is a mass spectrometer-based flow cytometer.

Elemental analysis can be used to detect the element tag and/or the element-tagged support. For example, where the element-tagged support is a coded bead, elemental analysis can be used to detect both the element tag and the element-tagged coded bead. Where the immobilized peptide substrate (e.g., the peptide substrate, or a first portion thereof, attached to an element-tagged support) has been separated from the biological medium, elemental analysis can be used to detect the element tag in the biological medium.

Elemental analysis can be used to provide a quantitative measurement of the element tag and/or the element-tagged support.

In certain embodiments, the biological medium is analyzed using particle elemental analysis. This method allows for accurate measurement of enzymatic activity without the need for separation of the tagged, immobilized peptide substrate. For example, a tagged, immobilized substrate that has not been cleaved (i.e., a tagged, immobilized peptide substrate that has not undergone proteolysis) will provide a signal that indicates the presence of both the element tag and the element-tagged support. Conversely, a tagged substrate that has been cleaved will provide a distinct and identifiable signal, corresponding to either the first portion (attached to the element-tagged support) or the second portion (attached to the element tag), as described above.

As a result, particle elemental analysis can be used to identify and quantify the presence of enzymatic activity at the level of individual particles. Furthermore. because a cleaved tagged peptide substrate provides a signal that is distinct and identifiable in comparison to an intact, unreacted tagged, immobilized peptide substrate, the process can be conducted in a single step, without the need for separation prior to analysis.

Generally, enzyme kinetics are characterized by the Michaelis constant K_{M}, and the maximum reaction rate, Vₘₐₓ. First, keeping a constant concentration of the enzymatic substrate, the rate of product formation can be determined by measuring the product concentration as a function of time. For example, tagged, immobilized peptide substrate on element-coded beads can be combined with a specific protease to form a reaction mixture, and aliquots withdrawn at specific time intervals (for example, 2 minutes, 4 minutes, 6 minutes. etc.) and processed as described above for elemental analysis of the cleaved products to obtain the rate of reaction V. Second, the rate of product formation can be determined for different initial tagged, immobilized peptide substrate concentrations at a constant protease concentration. Assuming a single-substrate protease kinetic reaction, the Michaelis-Menten equation can be used to determine K_{M} and Vₘₐₓ from the resulting data.

The methods described herein can be conducted in a multiplex format, in which the activities of multiple enzymes are measured simultaneously.

As used herein, a “multiplexed assay” refers to an assay in which multiple assay reactions (e.g., simultaneous, distinct reactions involving multiple analytes) are carried out in a single reaction chamber, and/or wherein multiple analytes are analyzed in a single detection step.

For example, in the incubation step described herein, a plurality of distinct tagged, immobilized peptide substrates can be incubated in the same biological medium. Each substrate can be tagged with a distinct element tag, and immobilized on a distinct element-tagged support. This allows each peptide substrate to be uniquely identified using elemental analysis (e.g., using mass cytometry).

The biological medium can further comprise a plurality of enzymes. Because each peptide substrate can be uniquely identified, through the presence of a distinct element tag, the activity of each corresponding enzyme (i.e., the enzyme specific to that substrate) can therefore also be quantitatively determined.

For example, the biological medium can comprise two or more peptide substrates that are substrates for two or more different protease enzymes.

Additionally, at least five different peptide substrates can be distinctly tagged and immobilized to form at least five distinct tagged, immobilized peptide substrates, where each peptide substrate is a substrate for a different protease enzyme. Each of the at least five element tags and element-tagged supports can be detected and quantified, for example, by mass cytometry.

Further, at least ten different peptide substrates can be distinctly tagged and immobilized to form at least ten distinct tagged, immobilized peptide substrates, where each peptide substrate is a substrate for a different protease enzyme. Each of the at least ten different element tags and element-tagged supports can be detected and quantified, for example, by mass cytometry.

Further, at least fifteen different peptide substrates can be distinctly tagged and immobilized to form at least fifteen distinct tagged, immobilized peptide substrates, where each peptide substrate is a substrate for a different protease enzyme. Each of the at least fifteen different element tags and element-tagged supports can be detected and quantified, for example, by mass cytometry.

Further, at least twenty different peptide substrates can be distinctly tagged and immobilized to form at least twenty distinct tagged, immobilized peptide substrates, where each peptide substrate is a substrate for a different protease enzyme. Each of the at least twenty different element tags and element-tagged supports can be detected and quantified, for example, by mass cytometry.

While keeping in mind that element tags can be different from the element-tagged supports, the maximum number of different peptide substrates that can be multiplexed in a single assay generally depends on the number of distinguishable element tags that can be simultaneously detected and quantified by mass cytometry. Moreover, in the similar fashion of employing various ratios of several different metals for the identification of different bead types, element tags for attaching to different peptide substrates can also be selected from the unique combination of different metals. Because the number of metals and their stable isotopes can be greater than 50, the number of different peptide substrates can be very large (over 10⁶ different element tags are feasible). However, it practice the operational limits of the mass cytometer and the data collection capabilities contribute to determining the maximum number of peptides substrates that can be simultaneously analyzed. Accordingly, it is reasonable to expect at least 100 different peptide substrates can be distinctly tagged and immobilized to form at least 100 distinct tagged, immobilized peptide substrates. More specifically, an upper range of 10 to 100 different peptide substrates can be distinctly tagged and immobilized to form at least 100 distinct tagged, immobilized peptide substrates.

The biological medium can further comprise a plurality of enzyme activators and/or inhibitors. For example, the biological medium can comprise two or more different enzyme inhibitors that are specific to two or more different enzymes.

The processes and methods described herein facilitate, for example, the preparation of efficient, accurate, and quantitative assays for gene reporter expression.

In biomedical and pharmaceutical research, reporter gene assays are widely used for the study of gene regulation and identification of factors that influence gene expression. Introduction of a reporter gene construct, which consists of one or more gene regulatory elements (i.e., sequence regions necessary for transcription of a functional mRNA together with a coding sequence for a reporter protein) into a live cell is followed by quantitation of the expressed protein or its enzymatic activity enabling an indirect measure of gene expression. Thus, a transfected beta-galactosidase reporter gene can indicate the presence of regulatory elements for a protein of interest in a given cell. The ability to detect multiple enzymes in a single aliquot of test sample, as described in the methods set forth herein, facilitates the identification of endogenous enzymes together with reporter enzyme activity (e.g., beta-galactosidase) encoded by a gene transfected into a cell.

An example of a specific application is the use of various kallikreins that are serine proteases as a prognostic marker for prostate cancer. Different kallikrein proteins are known to affect the prostate and these proteases have a known amino acid sequence. It has been proposed that the kallikrein protease concentration coupled with the prostate-specific antigen (PSA) concentration in serum can be used to determine the incidence of prostate cancer more accurately than measuring the PSA concentration alone and thereby decrease the number of prostatic biopsies. PSA is also a member of the kallikrein protease family.

Further, trypsin concentration in affected tissues can be used to determine the incidence of colorectal cancer (CRC). Trypsin activates, and is co-expressed with matrix metalloproteases (MMP-2, MMP-7, MMP-9 in CRC). Co-segregation of trypsin and MMPs within the minor environment is important for the activation of MMPs: this process can explain poor prognosis in colorectal cancer with increased trypsin concentrations. Together, trypsin and MMPs are particularly important in colorectal proliferation, progression, and invasion.

These are just two of the many various applications that the methods provided by the present disclosure can provide advantageous information regarding the activity of various enzymes in particular biological processes.

Further, the methods disclosed herein also facilitate the creation of substrate suspension arrays for high-throughput screening of enzymatic activity. Screening assays generally involve the comparison of two sets of reactions: a first set of reactions without the effector, and a second set of identical reactions except the effector is present. For example, biological media containing proteases and enzyme effectors of choice can be prepared at different concentrations of each reactant. Immobilized substrates on element-coded beads can then be added to each biological medium and incubated until the reactions are completed. The resulting mixtures are typically diluted to 10⁶ beads/mL, and the beads analyzed by mass cytometry. The data between reactions with and without enzyme effectors can then be compared.

As used herein, the term “support” refers to a solid surface to which the peptide substrate can be attached.

Non-limiting examples of supports include synthetic membranes, beads (e.g., elastomeric, agarose, silicate), and planar surfaces in plastic microwells, glass slides, and reaction tubes.

For example, the support can comprise a solid bead. The solid bead can comprise a polymeric, glass, or ceramic bead. The glass or ceramic bead can also comprise a metallic coating. The metallic coating can be comprised of a metal, metal alloy, or a combination thereof.

When the bead is a polymeric bead, the bead can comprise polystyrene, polymethyl-methacrylate, acrylonitrile, or a combination thereof. In certain embodiments, the bead comprises polystyrene.

The solid support can be a coded bead (i.e., a solid bead wherein one or more distinct elements are attached to and/or contained within the bead, thereby providing a distinct signal when interrogated by elemental analysis). For example, the support can comprise an element-stained bead as described in U.S. Application Publication No. 2010/10144056 A1, which is incorporated by reference in its entirety.

The coded bead can comprise two or more distinct staining elements. For example, the coded bead can comprise two or more distinct lanthanide elements.

In certain embodiments, the support comprises an element-stained bead wherein the staining elements uniformly diffused throughout the body of the bead.

Alternatively, the support comprises an element-stained bead wherein the staining elements penetrate said microsphere in a mariner that results in a distinct volume distribution of said staining elements.

The solid support can have a particle size of from about 0.1 micron to about 10 microns, from about 0.3 microns to about 10 microns, from about 0.5 microns to about 10 microns, from about 0.8 microns to about 10 microns, and from about 1 micron to about 10 microns.

As used herein, the term “element tag” refers to a chemical moiety that comprises an element or a plurality of elements attached to a supporting molecular structure, and which is distinguishable from the analyte and from other element tags on the basis of its elemental composition.

To be distinguishable from the analyte, an element tag can comprise one or more elements that are not present in the analyte, or which are present only in trace amounts. For example, the element tag can comprise a metal element.

In certain embodiments, the element tag can comprise a lanthanide element or a transition element. Non-limiting examples of typical lanthanide elements include europium, gadolinium, terbium, and ytterbium. The element tag can comprise a post-transition metal element selected from the group consisting of aluminum, gallium, indium, tin, thallium, lead, and bismuth.

Elemental analysis (e.g., mass cytometry) can be used to accurately detect and distinguish different isotopes of the same element. Accordingly, the element tag can be distinguishable on the basis of its isotopic composition. For example, the element tag can comprise a plurality of isotopes of an element.

An element tag is functionally distinguishable from a multitude of other element tags in the same sample because its elemental or isotopic composition is different from, that of the other tags.

The peptide substrate can be linked to the element tag or polymer element tag by standard linking moieties using standard chemistry that is well known to a person of ordinary skill in the art.

Where the element tag comprises a metal element, the element tag can also comprise one or more chelating groups.

The element tag can comprise a polymer carrier comprising a plurality of covalently attached chelating groups.

As used herein, the term “polymer” refers to a substance composed of molecules characterized by the multiple repetitious of one or more species of atoms or groups of atoms (constitutional units) linked to each other in amounts sufficient to provide a set of properties that do not vary markedly with the addition or removal of one or a few constitutional units. More generally, a polymer molecule can be described of in terms of its backbone, which is the connected link of atoms that span the length of the molecule, and the pendant groups, which are attached to the backbone portion of each constituent unit. The pendant groups can be chemically and functionally different from the backbone chain.

For example, the element tag can comprise a polymer, wherein the polymer comprises a plurality of pendant groups having a high affinity for metal ions, and which can act as chelating groups or ligands for those ions.

The element tag can be a polymer-based element tag as described in U.S. Application Publication No. 2008/0003616 A1, which is incorporated by reference in its entirety.

For example, the element tag can comprise a polymer, wherein the polymer comprises at least one metal-binding pendant group that comprises a diethylenetriaminepentaacetate (DTPA) ligand or a 1.4.7.1 0-tetraazacyclododecane-1.4.7.1 0-tetraacetic acid (DOTA) ligand, or an amide or ester thereof, and at least one metal atom.

The number of metal-binding pendant groups can be between about 10 and about 250. For example, the element tag can comprise a polymer comprising from about 10 to about 50 transition metal or lanthanide atoms. In certain embodiments, the polymer comprises from about 20 to about 50 transition metal or lanthanide atoms. Particularly, the polymer comprises from, about 25 to about 35 transition metal or lanthanide atoms.

The element tag comprises at least one transition or other metal atom. Alternatively, the element tag comprises at least one lanthanide atom.

The polymer can be selected from the group consisting of linear polymers, copolymers, branched polymers, graft copolymers, block polymers, star polymers, and hyperbranched polymers. For example, the backbone of the polymer can be derived from substituted polyacrylamide, polymethacrylate, or polymethacrylamide.

Generally, the processes and methods described herein can be used to detect the presence of any type of enzyme having an activity specific to a peptide substrate.

In certain embodiments, the enzyme is a protease, which broadly refers to any enzyme that conducts proteolysis. More particularly, a protease catalyzes hydrolysis of the peptide bonds that link together amino acids (e.g., amino acids that are linked in a peptide, polypeptide, or protein chain).

Non-limiting examples of proteases that can be identified in accordance with the methods disclosed herein include serine proteases, threonine proteases, cysteine proteases, aspartate proteases, glutamic proteases, and metalloproteases.

Non-limiting examples of serine proteases include trypsin, chymotrypsin, keratinase, plasmin, thrombin, fibrinolysin, collagenase, subtilisin, and elastase.

Non-limiting examples of cysteine proteases include calapins, cathepsins (A, B, and C), caspases, papain, and bromelain.

Non-limiting examples of aspartic proteases include pepsin, presenilin-1, presenilin-2, renin, gamma-secretase, plasmepsin, cathepsin-D, and cathepsin-E.

The processes and methods described herein can also be used to detect the presence of enzyme activators or inhibitors in the biological medium. For example, the activity of enzyme activators or inhibitors can be detected using a screening assay as described above, in which data from reactions with a prospective activator or inhibitor are compared with data from otherwise identical reactions wherein the activator or inhibitor is not present.

As used herein, the term “peptide substrate” generally refers to a molecule comprising two or more amino acids linked by peptide bonds.

For example, the peptide substrate can comprise a peptide, a polypeptide, or a protein. As understood by one skilled in the art, the size boundaries between peptides, polypeptides, and proteins are not fixed, and as used herein, the terms interchangeably refer to compounds comprising two or more amino acids joined by one or more covalent chemical bonds.

The substrate can be a synthetic or naturally occurring entity.

Non-limiting examples of naturally occurring peptide substrates include proteins such as hemoglobin, myoglobin, spectrin, fibronectin, collagen, keratin, elastin, gelatin, insulin, and albumin.

The first amino acid and the last amino acid of the peptide substrate can be a C-terminal amino acid or a N-terminal amino acid. When the first amino acid of the peptide substrate is a C-terminal amino acid, the last amino acid of the peptide substrate is a N-terminal amino acid. Conversely, when the first amino acid of the peptide substrate is a N-terminal amino acid, the last amino acid of the peptide substrate is a C-terminal amino acid.

The biological medium can further comprise one or more additional components.

For example, the biological medium can further comprise a candidate effector of enzyme activity. As used herein, the term “effector” refers to a molecule that is capable of increasing or decreasing the activity of an enzyme, directly or indirectly. As used herein, the term “candidate effector” refers to a molecule which can act to increase or decrease the activity of an enzyme.

Non-limiting examples of effectors include macromolecules, such as proteins, glycoproteins, polysaccharides, glycosaminoglycans, proteoglycans, integrins, enzymes, lectins, selectins, cell-adhesion molecules, toxins, bacterial pili, transport proteins, hormones, antibodies, major histocompatability complexes, immunoglobulin superfamilies, and cadherins. Additional non-limiting examples of effectors include small molecules, such as putative drugs, monosaccharides, disaccharides, oligosaccharides, amino acids, oligopeptides, nucleosides, nucleotides, oligonucleotides, lipids, retinoids, steroids, and glycopeptides.

The biological medium, can further comprise one or more internal standards. Generally, an internal standard refers to a known amount of a compound, distinct from the analyte that is added to the biological medium.

The addition of an internal standard to the biological medium is particularly useful when mass spectrometry is employed as the analytical method. When the biological medium is analyzed, the signal obtained from the internal standard can be used to calibrate the analytical results. For example, by comparing the strength of the mass spectrometric signal from the analyte with that of the internal standard, which is present in a known quantity, a quantitative measurement of the analyte can be derived.

Having described the invention in detail, it will be apparent that modifications and variations are possible without departing from the scope of the invention defined in the appended claims.

### EXAMPLES

The following non-limiting examples are provided to farther illustrate the present invention.

### Example 1

### Particle Elemental Analysis Using a Mass Cytometer

Four orthogonal peptide substrates are synthesized for the proteases calpain-1, caspase-3, MMP-9 and ADAM10. Each substrate carries a biotin tag at the C-terminus and a DTPSA-based lanthanide complex at the N-terminus. The proteases belong to four different families of enzymes and play important roles in normal physiological processes as well as in various diseases. For example, matrix metalloproteinase-9 (MMP-9) is essential in the restructuring of extracellular matrix during bone development and wound healing, and is also involved in cancer metastasis and arthritis, ADAM10, as a further example, is essential in the proteolytic processing of amyloid precursor protein to form the beta amyloid, which is deposited in amyloid plaques found in the brains of Alzheimer's patients.

Each peptide substrate is then immobilized through attachment to a unique coded bead having a thiol-functionalized surface. Each coded bead comprises polystyrene stained with a distinct proportion of two lanthanide elements, and therefore provides a distinct signal when interrogated using mass cytometry.

Each immobilized substrate is then tagged with an element tag, wherein each element tag comprises transition metal or lanthanide. The element tag provides a distinct signal when interrogated using elemental analysis.

The tagged, immobilized peptide substrates are then incubated in a vessel comprising a biological medium, which comprises a sample obtained from a HeLa cell lysate. The substrates are incubated with the sample for 2 hours at approximately 25° C.

The biological medium is then interrogated by mass cytometry. As each particle passes through the mass cytometer, the resulting mass spectrometric signal can be used to quantify the status of the peptide substrate. For example, the detection of the transition metal or lanthanide element corresponds to the presence of a particular element tag. If this signal coincides with a second signal indicating the presence of the corresponding lanthanide-stained coded bead support, it is inferred that the substrate is still intact, and has not undergone proteolysis. Conversely, if no signal is detected corresponding to the coded bead, it is inferred that the detected particle is the second portion (i.e., the element-tagged portion) of a substrate that has been cleaved by proteolysis. In this manner, a quantitative analysis of the enzymatic activity in the sample is obtained.

For example, kallikrein can be used as a prognostic marker for prostate cancer. Various kallikrein genes encode kallikrein enzymes that are proteases: these proteases have a known amino acid sequence. The substrates of these kallikrein proteases are also known. Thus, the known peptide substrate of one or more of the kallikrein proteases can have a coded bead attached to the C-terminal or the N-terminal amino acid of the peptide and the other amino acid of the peptide substrate can be attached to an element tag or element-tagged polymer using methods known in the art. Once the peptide substrate is immobilized through attachment to the coded bead and tagged with an element tag, it can be incubated with the biological sample of interest and interrogated by mass cytometry. The mass cytometry data can provide information regarding the activity of the specific kallikrein proteases and allow inferences regarding the kallikrein concentration. It has been proposed that a combination of the kallikrein protease concentration and prostate-specific antigen (PSA) concentration in serum can be used to determine the incidence of prostate cancer more accurately than measuring the PSA concentration alone. This data can potentially decrease the number of prostatic biopsies.

Additionally, the trypsin concentration can be used to determine the incidence of colorectal cancer (CRC). Trypsin activates, and is co-expressed with matrix metalloproteases (MMP-2, MMP-9 in CRC). Co-segregation of trypsin and MMPs within the tumor environment is important for the activation of MMPs. The S substrates for trypsin and MMPs are known and can be coded and tagged as described herein to provide an immobilized, tagged peptide substrate. Once the substrate is immobilized and tagged, it can be interrogated by mass cytometry to provide information about the activity of the targeted enzyme activity and concentration. Collection of the data can explain the poor prognosis observed for colorectal cancer in patients having increased trypsin concentrations in the affected tissues. Together trypsin and MMPs are particularly important in colorectal proliferation, progression, and invasion, so knowing the activity of these proteases in specific tissues can provide additional information regarding the progression of colorectal cancer.

When introducing elements of the present invention or certain, embodiments(s) thereof the articles “a”. “an”. “the” and “said” are intended to mean that there are one or more of the elements. The terms “comprising”, “including” and “having” are intended to be inclusive and mean that there can be additional elements other than the listed elements.

In view of the above, it will be appreciated that the several objects of the invention are achieved and other advantageous results obtained.

As various changes can be made in the above products and methods without departing from the scope of the invention, it is intended that all matter contained in the above description and shown in the accompanying drawings shall be interpreted as illustrative and not in a limiting sense.

1. A method for determining protease activity in a biological fluid, the method comprising
attaching a coded bead to a first amino acid of a peptide substrate to form an immobilized peptide substrate, the peptide substrate comprising the first amino acid and a last amino acid and being a substrate for a protease enzyme:
attaching an element tag to the last amino acid of the peptide substrate to form an immobilized tagged peptide substrate:
incubating the immobilized, tagged peptide substrate with a biological fluid:
detecting the element tag and the coded bead in the biological fluid by elemental analysis: and
determining protease activity within the biological fluid based on detecting the element tag.

2. The method of claim 1, wherein detecting the element tag and the coded bead comprises using mass cytometry.

3. The method of claim 1 or 2, wherein the peptide substrate comprises two or more peptide substrates, wherein the two or more peptide substrates are substrates for two or more different protease enzymes.

4. The method of any one of claims 1 to 3, wherein detecting the element tag and the coded bead comprises quantifying the elemental tags using mass cytometry.

5. The method of any one of claims 1 to 4, further comprising separating the immobilized peptide substrate from the biological fluid to form an analyte solution and detecting the element tag.

6. The method of claim 5, wherein the element tag is detected by mass cytoinetry.

7. The method of any one of claims 1 to 6, wherein the coded bead comprises a unique element or a unique combination of elements for the coded bead.

8. The method of any one of claims 1 to 7, wherein the first amino acid of the peptide substrate is a C-terminal amino acid.

9. The method of any one of claims 1 to 7, wherein the first amino acid of the peptide substrate is a N-terminal amino acid.

10. The method of any one of claims 1 to 8, wherein the last amino acid of the peptide substrate is a N-terminal amino acid.

11. The method of any one of claims 1 to 7 and 9, wherein the last amino acid of the peptide substrate is a C-terminal amino acid.

12. The method of any one of claims 1 to 11, wherein the element tag comprises a transition metal element.

13. The method of any one of claims 1 to 11, wherein the element tag comprises a post-transition metal element selected from the group consisting of aluminum, gallium, indium, tin, thallium, lead, and bismuth.

14. The method of any one of claims 1 to 11, wherein the element tag comprises a lanthanide element.

15. The method of any one of claims 1 to 14, wherein the element tag is attached to a polymer comprising 10 to 100 transition metal, post-transition metal, or lanthanide atoms.

16. The method of claim 15, wherein the element tag is attached to a polymer comprising 20 to 50 transition metal, post-transition metal, or lanthanide atoms.

17. The method of claim 15, wherein the element tag is attached to a polymer comprising 25 to 35 transition metal, post-transition metal, or lanthanide atoms.

18. The method of any one of claims 1 to 17, wherein five or more peptide substrates are substrates for five or more different protease enzymes.

19. The method of any one of claims 1 to 17, wherein ten or more peptide substrates are substrates for ten or more different protease enzymes.

20. A method for detecting protease activity in a biological fluid, wherein the method comprises:
attaching a coded immobilization moiety to a first amino acid of at least five different peptide substrates to form at least five different coded immobilized peptide substrates, wherein each of the at least five different peptide substrates is a substrate for a different protease enzyme:
attaching a different element tag to the last amino acid of each of the at least five different coded immobilized peptide substrates to form at least five different immobilized tagged peptide substrates:
incubating the at least five different immobilized tagged peptide substrates with a biological fluid: and
detecting the element tags and the coded immobilization moieties in the biological fluid by mass cytometry: and
determining the protease activity in the biological fluid based on detecting the elemental tags and the coded immobilization moieties.

21. The method of claim 20, further comprising separating the immobilized peptide substrates from the biological fluid to form an analyte solution and detecting the different element tags in the analyte solution by mass cytometry.

22. The method of claim 20 or 21, wherein detecting the element tag and the coded immobilization moiety comprises using mass cytametry.

23. The method of any one of claims 20 to 22, wherein the coded immobilization moiety is a coded bead and comprises a unique element or a unique combination of elements.

24. The method of any one of claims 20 to 23, wherein the first amino acid of each of the peptide substrates is a C-terminal amino acid.

25. The method of any one of claims 20 to 24, wherein the first amino acid of each of the peptide substrates is a N-terminal amino acid.

26. The method of any one of claims 20 to 24, wherein the last amino acid of each of the peptide substrates is a N-terminal amino acid.

27. The method of any one of claims 20 to 23 and 25, wherein the last amino acid of each of the peptide substrates is a C-terminal amino acid.

28. The method of any one of claims 20 to 27, wherein the element tag comprises a transition metal element.

29. The method of any one of claims 20 to 27, wherein the element tag comprises a lanthanide element.

30. The method of any one of claims 20 to 27, wherein the element tag comprises a post-transition metal element selected from the group consisting of aluminum, gallium, indium, tin, thallium, lead, and bismuth.

31. The method of any one of claims 20 to 30, wherein the element tag is attached to a polymer comprising 10 to 100 transition metal, post-transition metal, or lanthanide atoms.

32. The method of claim 31 wherein the element tag is attached to a polymer comprising 20 to 50 transition metal, post-transition metal, or lanthanide atoms.

33. The method of claim 31, wherein the element tag is attached to a polymer comprising 25 to 35 transition metal, post-transition metal, or lanthanide atoms.

34. The method of any one of claims 20 to 33, wherein the at least five peptide substrates comprises at least 10 peptide substrates, wherein each of the at least 10 peptide substrates is a substrate for a different protease enzyme.
